(19) United States
(12) Patent Application Publication
(10) Pub. No.: US 2005/0255980 A1
(43) Pub. Date: Nov. 17, 2005

(54) Sealed package capable of sterilization

(76) Inventors: Steven Thomas Ventura, The Colony, TX (US); John B. Ristuccia, McKinney, TX (US)

Correspondence Address: FISH & RICHARDSON P.C., 1717 MAIN STREET, SUITE 5000, DALLAS, TX, 75201, US

(21) Appl. No.: 10/842,811

(22) Filed: May 11, 2004

(51) Int. Cl. A61L 2/00

(52) U.S. Cl. 493/197

(57) ABSTRACT

A system and method for manufacturing a sterilizable package includes cutting a vent in a portion of a top-web, cutting a vent-cover of porous vent media (PVM) from a PVM web. The vent-cover is sized larger than the vent, and the porous vent media is adapted to permit sterilization there through. The method also includes sealing the vent-cover to the top-web using heat, pressure, and time to cover the vent and sealing the portion of the top-web to a portion of a bottom-web using heat and pressure over a period of time. Sealing the top-web to the bottom-web creates a sealed package perimeter, and the vent-cover is within the sealed package perimeter.

### TECHNICAL FIELD

The present invention relates generally to sealed packages, and more specifically to a system and method for manufacturing sealed packages capable of sterilization.

### BACKGROUND

Many medical facilities and medical professionals require the use of sterilized medical disposable devices (DD). Due to the increasing cost of on-site sterilization, medical supply companies often provide DD to hospitals, clinics, doctors and other medical professionals that are pre-sterilized. Though packaging medical DD in a sterilized environment is often difficult and cost-prohibitive, many suppliers and packaging companies produce medical DD in packages designed to enable sterilization of the DD after packaging. This post-packaged sterilization may be performed at a hospital or other medical facility after receiving the packaged products, or the sterilization may be performed by the packager, distributor, or other third party intermediary.

Generally, medical packages that allow for post-packaging sterilization are manufactured with a thermoplastic layer sealed to a fibrous layer. Generally, a package may be created by forming a series of cavities in a continuous web of thermoplastic bottom-stock. After the medical DD has been placed in the cavity, the package is sealed by placing a layer of fibrous material over the cavity and sealing the medical DD between the two layers. The fibrous material, such as Du Pont's Tyvek™ or other suitable porous vent media (PVM), allows for sterilization by a variety of techniques, such as Ethylene Oxide (ETO) sterilization or other steam sterilization techniques, after the package is sealed.

As the cost of PVM sheets, such as Tyvek™ increases, the associated cost of producing sterilizable medical packaging increases. Accordingly, there exists a need for a sterilizable package that is more cost efficient than current sterilizable packages.

### SUMMARY

In one general aspect, a method for manufacturing a sterilizable package includes cutting a vent in a portion of a top-web, cutting a vent-cover of porous vent media (PVM) from a PVM web. The vent-cover is sized larger than the vent, and the porous vent media is adapted to permit sterilization there through. The method also includes sealing the vent-cover to the top-web using heat, pressure, and time to cover the vent and sealing the portion of the top-web to a portion of a bottom-web using heat and pressure over a period of time. Sealing the top-web to the bottom-web creates a sealed package perimeter, and the vent-cover is within the sealed package perimeter. The PVM web may be a polyolefin, polyethelene, or paper web. Additionally, the top-web may be introduced in a first direction and the PVM web may be introduced in a second direction, and the second direction may be substantially normal to the first direction.

In another general aspect, a system for manufacturing a sterilizable package includes a top-web and a vent cutting system adapted to create a vent in the top-web. A porous vent media (PVM) web adapted to allow gases to pass there through and to substantially reduce the passage of bacteria there through is also included. A vent-cover cutting system is adapted to remove a vent-cover from the PVM web, so that the removed vent-cover has dimensions slightly greater than the vent in the top-web. A first sealing system seals the vent-cover to the top-web, so that the vent-cover is sealed around the vent in the top-web. A second sealing system seals a portion of the top-web to a portion of a bottom-web, such that the top-web sealed to the bottom stock web forms an enclosed space, and the enclosed space is bounded by the sealed portions of the top-web and bottom-web. The top-web may be a thermoplastic material, and the bottom-web may be a web of polymer fibers. The vent-cover cutting system may include a drive motor that is operable to draw the PVM web into a cutting area, a transfer block operable to transfer the vent-cover to the first sealing system, a blade adapted to remove the vent-cover from the PVM web; and a tamp-pad adapted to maintain the vent-cover in position on the transfer block when the blad removes the vent-cover from the PVM web.

Various implementations of the present invention provide desirable features. For example, using a vent-cover sealed to a package reduces the amount of PVM material required to manufacture a sterilizable package. Additionally, systems manufactured according to implementations reduce waste associated with cutting PVM vent-covers for manufacturing sterilizable packaging. Yet another feature is a reduction in malfunctions due to undesired creasing, folding, or improper positioning of the PVM web during cutting operations.

The details of one or more implementations are set forth in the accompanying drawings and the description below. Other features will be apparent from the description and drawings and from the claims.

### DESCRIPTION OF DRAWINGS

FIG. 1A is a side view of an apparatus for manufacturing sterilizable packages.

FIG. 1B is a perspective view of the apparatus illustrated in FIG. 1A.

FIG. 1C is a plan view of a portion of the apparatus illustrated in FIGS. 1A and 1B.

FIG. 2A is a side view of an apparatus for cutting a piece of a porous vent medium.

FIG. 2B is a perspective view of the apparatus illustrated in FIG. 2A.

FIG. 3 is a perspective view of an apparatus for sealing a vent-cover to a web of packaging material.

FIG. 4 is a perspective view of an apparatus for cutting a vent in a web of packaging material.

FIG. 5 is a flow diagram of a method according to an implantation of the present invention.

Like reference symbols in the various drawings indicate like elements.

### DETAILED DESCRIPTION

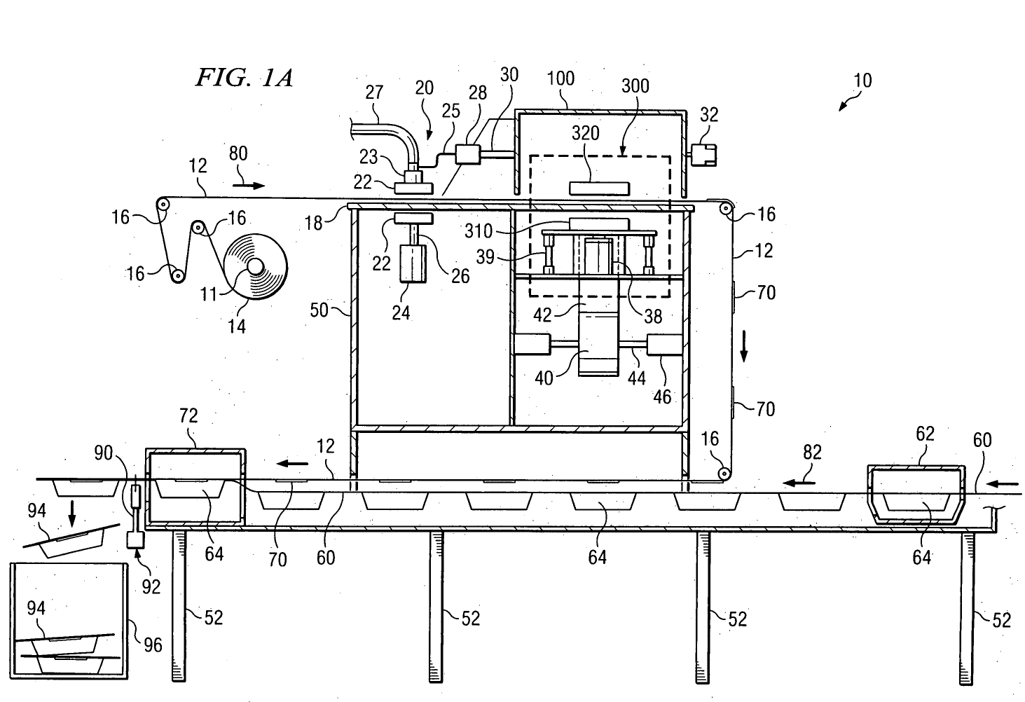

The present invention alleviates a significant amount of cost and waste associated with current methods and systems for manufacturing sterilizable packages. Referring to FIGS. 1A, 1B, and 1C, an apparatus 10 for manufacturing a sterilizable package includes a top-stock web or “top-web” 12 drawn from top-stock roll 14 mounted on a mandrel 11. The top-web 12 may be of any suitable packaging material. For example, the top-web 12 may be thermoplastic, a polymer blend, or other suitable packaging material.

The top-web 12 is drawn over rollers 16. Rollers 16 may include a single roller 16 or multiple rollers 16. The top-web 12 is drawn through the apparatus 10 along a vent-cutting direction 80 and over a platen 18 to a vent cutter 22. In one implementation, the platen 18 includes vacuum ports 19. The vacuum ports 19 are adapted to provide vacuum pressure on the top-web 12 to maintain the top-web on the platen 18 and to prevent undesired folding or movement of the top-web 12 in a direction other than the vent-cutting direction 80. As the top-web 12 is drawn through the vent cutter 22, the vent cutter 22 removes a portion of the top-web material by punching or cutting the top-web 12 to create a vent 76 in the top-web. In the implementation shown, the vent cutter 22 punches through a portion of the top-web 12 to completely remove the punched piece of the web from the top-web 12 to create a vent 76 in the top-web 12. Alternatively, though not illustrated, the vent-cutter may operate with an automated blade that cuts a pre-determined shaped vent in the top-web 12.

In the implementation shown, the vent cutter 22 removes a portion of top-web material from the top-web 12 so that the vent 76 has a length and a width, where the length is measured substantially along the vent-cutting direction 80. Alternatively, the vent-cutter 22 could create a vent with the length measured substantially normal to the vent-cutting direction 80. Additionally, the vent-cutter 22 could cut a vent 76 in any suitable orientation with respect to the top-web 12 and/or the vent-cutting direction 80.

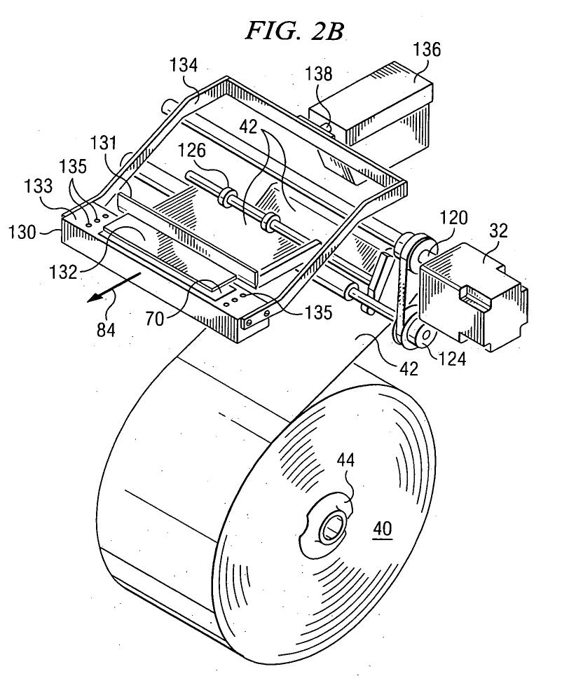

The material removed from the top-web 12 may be disposed through a vacuum tube 27. In operation, a pneumatic actuator 24 may actuate the vent cutter 22 by a push rod 26 coupled to the vent cutter 22. The vent cutter 22 according to the implementation shown is explained in more detail below with respect to FIG. 4. Alternatively, the vent cutter 22 may be actuated manually, by an electric actuator, or by other suitable actuator (not illustrated). After the vent 76 is removed from the top-web 12, the top-web 12 is indexed along the vent-cutting direction 80 to a vent-cover system 100.

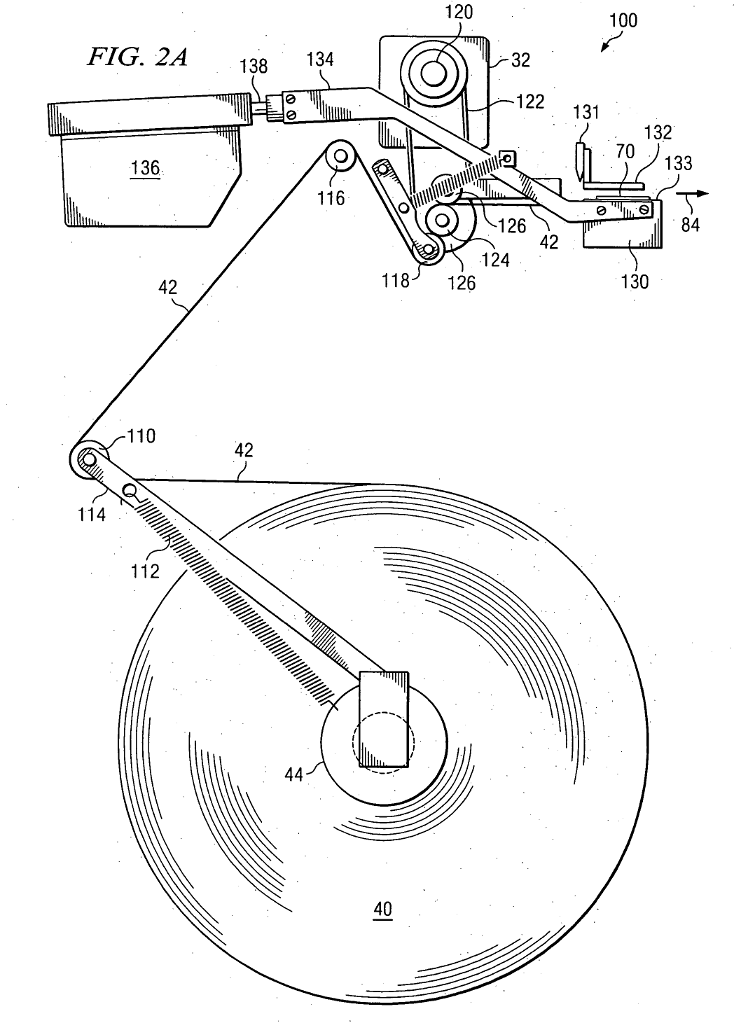

At vent-cover system 100, a web of porous vent media (PVM) or PVM web 42 is drawn from a PVM roll 40 mounted on a PVM mandrel 44 in a transfer direction 84. As shown, the transfer direction 84 is substantially normal to the longitudinal axis of the vent 76. However, the transfer direction is relevant only to the extent that the vent-cover 70 that is cut by the vent-cover system 100 and transferred to the sealing system 300 (described in more detail below with respect to FIG. 3) has a length that is measured substantially along the longitudinal axis of the vent 76. The vent-cover 70 may be of any number of dimensions. Generaly, the dimensions of the vent-cover 70 are sufficient to completely cover the vent 76 in the top-web 12. Additionally, the PVM web may be any suitable PVM that allows for sterilization through current or future technology. Accordingly, the PVM web 42 may be a polyethylene or polyolefin fiber web such as Tyvek™ made by Du Pont™, another polymer-fiber web material that allows sterilization through steam sterilization or ethylene oxide sterilization, such as cellulose, paper, or any other material now-known or developed hereafter suitable for sterilizable packaging.

After the vent-cover system 100 cuts the vent-cover 70, the vent-cover 70 is transferred to a sealing system 300, which is discussed in greater detail below with respect to FIG. 3. In the implementation shown, the top-web 12 is indexed through the apparatus 10 over the platen 18 in the vent-cutting direction 80 until the vent 76 is positioned between a support brille 320 and the a sealing plate 310 of the sealing system 300. The vent-cover 70 is retained on the support brille 320. The support brille 320, with the vent-cover 70 retained on the support brille face 322, is biased toward the top-web 12 until the vent-cover 70 is in contact with the top-web 70. The sealing plate 310, which may be positioned on the opposite side of the top-web 12 from the support brille 320, is also biased toward the top-web 12. The sealing plate 310 and/or the support brille 320 may also be heated.

When the sealing plate 310 contacts the top-web 12 around the vent 76, and the support brille 320 is on the opposite side of the top-web 12, the vent-cover 70 is between the support brille 320 and the heated sealing plate 310. In this implementation, the heat of the sealing plate 310 and the pressure exerted on the top-web 12 and the vent-cover 70 by the support brille 320 and the sealing plate face 312 operate jointly through heat, pressure, and time to affix the vent-cover 70 to the top-web 12. In the implementation shown, the vent-cover 70 is sealed around the edges of the vent 76 so that the vent-cover 70 completely covers the vent 76. After the vent-cover is affixed to the top-web 12, the top-web is indexed to a package sealing system 72, where the top-web 12 with vent-covers 70 affixed to vents 76 is sealed to a bottom web 60 drawn through the apparatus 10 along a production direction 82.

The bottom-web 60 may be of a suitable packaging material such as a thermoplastic or other suitable material. Compartments 64 may be formed in portions of the bottom-web 60 by a forming system 62. The compartments 64 may be used to retain items (not illustrated) to be packaged by the apparatus 10. As the top-web 12 is indexed through the apparatus 10, it progresses around the rollers 16 until it is positioned adjacent to the bottom-web 60. In the implementation shown, the vent-covers 70 are positioned adjacent to the compartments 64. The top-web 12 and the bottom-web 60 are indexed together in the direction of production 82 toward and through the package sealing system 72. In the implementation shown, the package sealing system 72 operates to seal the top-web 12 to the bottom-web 60 by creating a sealed perimeter 78 around the compartment 64. The vent-cover 70 may be either sealed to the outside of the top-web 12 or the inside of the top-web 12 relative to the compartment 64. Accordingly, a vent-cover 70 sealed to the “outside” of the top-web 12 is a vent-cover 70 that is not within the compartment 64 bounded by the top-web 12 sealed around the compartment 64. Alternatively, a vent-cover 70 sealed to the “inside” of the top-web 12 is positioned between the top-web 12 and the bottom-web 60.

After the top-web 12 is sealed to the bottom-web 60, a sterilizable package 94 is produced. Upon exiting the package sealing system 72, a cutting system 92 may cut the sterilizable package 94 from the top-web 12 and the bottom-web 60 such that the sterilizable package 94 is a discrete unit. Alternatively, the sterilizable package 94 may remain attached to multiple other sterilizable packages 94 such that a group of sterilizable packages 94 remain connected to each other (e.g. by perforating the top-web 12 and the bottom-web 60 between the various sterilizable packages 94).

Each of the systems explained above may be mounted to a frame 50 having supports 52. Additionally, a container 96 may be implemented to retain the sterilizable packages 94 manufactured by the apparatus 10.

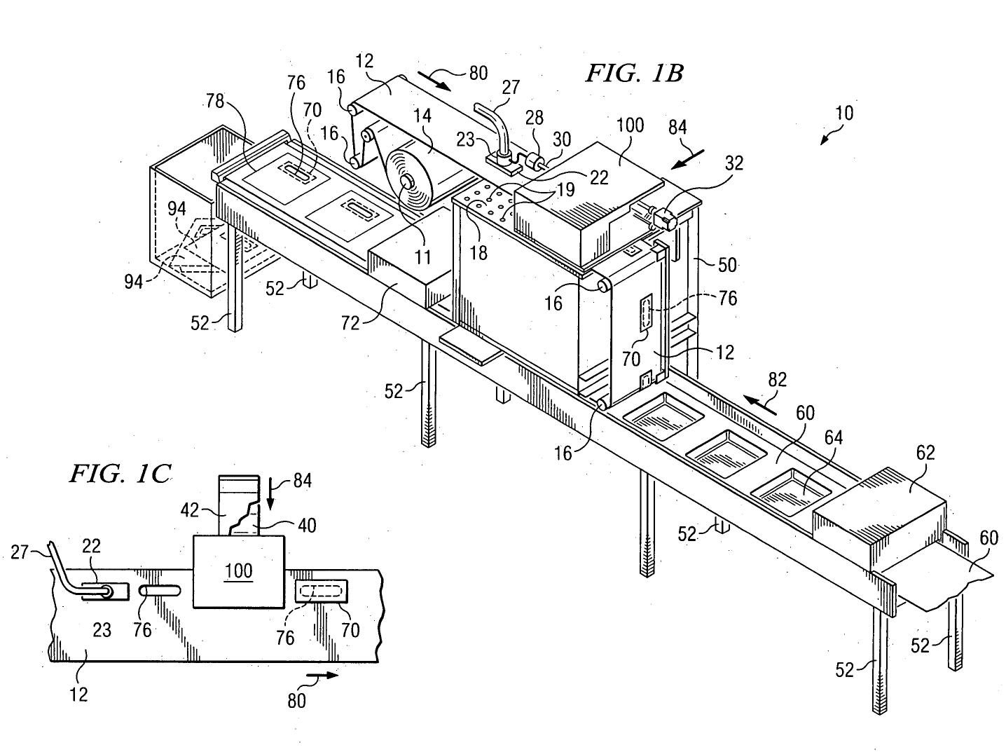

FIGS. 2A and 2B illustrate the vent-cover system 100 in greater detail. In the implementation shown, the PVM roll 40 is mounted on a mandrel 44. A PVM web 42 is drawn from the PVM roll 40 around a guide roller 110. The guide roller 110 is coupled to the mandrel 44 by a spring tensioner 114 having a spring 112 (FIG. 2A). The spring tensioner maintains the orientation of the PVM web 42 to ensure that the PVM web 42 is free of folds and/or wrinkles as the PVM web 42 progresses through the vent-cover system 100.

The PVM web 42 progresses around the guide roller 110 and around an idle roller 116. The idle roller 116 may not be required, but in certain implementations the idle roller 116 may be preferable to control the angle at which the PVM web 42 is pulled through the system by the drive roller 124. A drive shaft 120 rotates that drive roller 124 by exerting a rotational force on a drive belt 122. The drive shaft 120 is rotated by an electric motor 32. Alternatively, though not illustrated, any suitable drive mechanism may be substituted for the electric motor 32, drive shaft 120, drive roller 124, and drive belt 122. For example, a sprocket system or chain-driven system may be used. In yet another implementation, the drive roller 124 may be directly rotated by the electric motor. In still another implementation, the drive roller 124 may be rotated by a pneumatic actuator, a hydraulic actuator, or other suitable drive system.

A pinch roller 118 may be positioned adjacent to the drive roller 124 to provide sufficient friction between the PVM web 42 and one or more friction rollers 126 of drive roller 124 to force the PVM web 42 onto a transfer block 130. The transfer direction 84 is illustrated as substantially normal to the vent-cutting direction 80. This orientation, though not necessary, may prevent unwanted folds or problems arising with the PVM web 42, because more force is necessary to bend or fold the PVM web 42 when a smaller length of the PVM web 42 is drawn through the drive roller 124 and the pinch roller 118. Thus, the smaller the amount of the PVM web 42 that is introduced through the drive roller 124 and the pinch roller 118, the less likely the portion of the PVM web 42 that is in place on the transfer block 130 will fold or bend prior to, or during, cutting of the vent-cover 70. Also, the smaller width of the vent-cover 70 allows more vent-covers 70 to be cut from a single PVM roll 40 before changing the PVM roll 40 relative to a PVM roll 40 that has a like-length of PVM material. Additionally, the fact that the vent 76 has a length corresponding substantially to the vent-cutting direction 80, along with the orientation of the PVM web 42, allows the vent 70 to be cut normal to the direction of the transfer direction 84. This transverse orientation allows the vent-cover 70 to be properly oriented and cut from the PVM web 42 without the need for rotating the top-web 12 or the vent-cover 70 prior to sealing the vent-cover 70 to the top-web 12 over the vent 76.

Alternatively, by simply rotating the vent-cutter 22 by 90 degrees, the vent-cover system 100 may also be rotated 90 degrees such that the vent-cover 70 is oriented substantially the same as the vent 76.

Once a sufficient amount of the PVM web 42 is present on the transfer block 130, a tamp pad 132 is biased onto the transfer block face 133 to hold the PVM web 42 in place. In one implementation, the transfer block face 133 includes vacuum ports 135 to further ensure the PVM web 42 is stationary for cutting. After the tamp pad 132 is in contact with the PVM web 42 to maintain the position of the PVM web 42 on the transfer block face 133, a blade 131 is biased to the PVM web 42 at the edge of the transfer block to cut the vent-cover 70 from the PVM web 42. If the PVM is unsuccessfully held by the vacuum ports 135 on the transfer block face 133, the tamp pad 132 may be automatically biased onto the vent cover 70 to allow the vacuum ports 135 to engage the vent cover 70 to hold the vent cover 70 on the transfer block face 133. After the vent-cover 70 is cut from the PVM web 42, the tamp pad 132 may be biased away from the transfer block face 133 so that the vent-cover 70 can be moved into position to be sealed onto the vent 76 cut in the top-web 12 (illustrated in FIGS. 1A and 1B).

Referring now to FIG. 3, in addition to FIGS. 2A and 2B, vent-cover system 100 operates with vent sealing system 300 to prepare the vent-cover 70 to be affixed to the top-web 12 (illustrated in FIGS. 1A and 1B). The transfer block 130 with the vent-cover 70 held on the transfer block face 133 is moved along the transfer direction 84 until it is between the support brille 320 and the sealing plate 310. The transfer block 130 may be moved by any suitable means. In the implementation shown, the transfer block 130 is coupled to an actuator 136 by a transfer frame 134. An actuating rod 138 may also be coupled to the frame 134 (FIG. 2A) so that when the actuator 136 is engaged, the actuating rod 138 forces the transfer block 130 along transfer direction 84 to a location adjacent to the support brille 320 for transfer of the vent-cover 70 from the vent-cover system 100 to the vent sealing system 300.

After the transfer block 130 is in place adjacent to the support brille 320, the support brille 320 may be biased toward the vent-cover 70. The support brille 320 includes a support brille face 322 and may also include vacuum ports 324. In the implementation shown, the vent-cover 70 is transferred to the support brille face 322 by activating the vacuum ports 324 on the support brille face 322 and deactivating the vacuum ports 135 on the transfer block face 133 of the transfer block 130. The vacuum ports 135 may be simultaneously reversed upon activation of the vacuum ports 324 on the support brille face 322 to clear residual suction. Additionally, or alternatively, the transfer block 130 may be biased toward the support brille 320 to ensure a transfer of the vent-cover 70. In an alternate implementation, the transfer block 130 and/or the support brille 320 may be pre-positioned with a small space between them when the transfer block 130 is moved into position for transfer along transfer direction 84 so that neither the support brille 320 nor the transfer block 130 must be biased toward each other for a transfer of the vent-cover 70.

If the transfer of the vent-cover 70 from the transfer block 130 to the support brille 320 is unsuccessful, the system may automatically reattempt the transfer. A sensor 326 may monitor the transfer of the vent-cover 70. In one implementation, the sensor may be coupled to the vacuum ports 324 of the support brille 320 so that if the vent-cover 70 is not transferred to the support brille face 322, the system automatically determines that the vacuum pressure was unchanged and reattempts the transfer. Alternatively, an optical sensor, not shown, may be included to detect the transfer of the vent-cover 70 from the transfer block 130 to the support brille face 322.

After the vent-cover 70 is transferred from the transfer block 130 to the support brille 320, the transfer block 130 may be removed from between the support brille 320 and the sealing plate 310. The sealing plate 310 may be coupled to a bracket 302 affixed to the frame 50 as illustrated in FIG. 1A. A seal plate actuator 318 may be used to bias the seal plate face 312 toward the top-web 12 when the vent 76 is positioned between the seal plate 310 and the support brille 320. The seal plate face 312 may be pre-heated so that when the seal plate face 312 contacts the top-web 12 around the vent 76, the portion of the top-web 12 in contact with the seal plate face 312 is melted or softened when the vent-cover 70 is placed in contact with the top-web 12 for faster sealing of the vent-cover 70. The support brille 320 with the vent-cover 70 attached to the support brille face 322 is also brought into contact with the top-web 12 around the vent 76. The pressure of the support brille face 322 pressed against the seal plate face 312 for a specified amount of time, in addition to the heat generated by the seal plate face 312, operate to sufficiently melt the top-web 12 and vent-cover 70 so that the vent-cover is affixed to the top-web 12 to cover the vent 76.

Referring now to FIG. 4, the vent cutter 22 includes an upper clamp 410 and a lower clamp 412. The lower clamp 412 is affixed to a push-plate 436. A spring 434 is disposed between the lower clamp 412 and the push-plate 436. A blade 420 is coupled to the push-plate 436. A vent cutter driver 430 is coupled to the push-plate 436 by a piston 432. During operation, top-web 12 is indexed between the upper clamp 410 and the lower clamp 412. To cut a vent 76 in the top-web 12, the piston 432 pushes the push-plate 436 toward the upper clamp 410. The lower clamp 412, which is also coupled to the push-plate 436, is biased toward the top-web 12. The lower clamp 412 engages the top-web 12 against the upper clamp 410 to help prevent ripping or tearing of the top-web 12. The push-plate 436 continues toward the lower clamp 412 after the lower clamp 412 has engaged the top-web 12 with the upper clamp 410. The continued motion of the push-plate 436 toward the top-web 12 forces the blade 420 through the top-web 12 to create the vent in the top-web 12. The spring 434 exerts a force against the push-plate 436, and therefore the blade 420 from the lower clamp 412 to assist the push-plate 436 to return to its unbiased position.

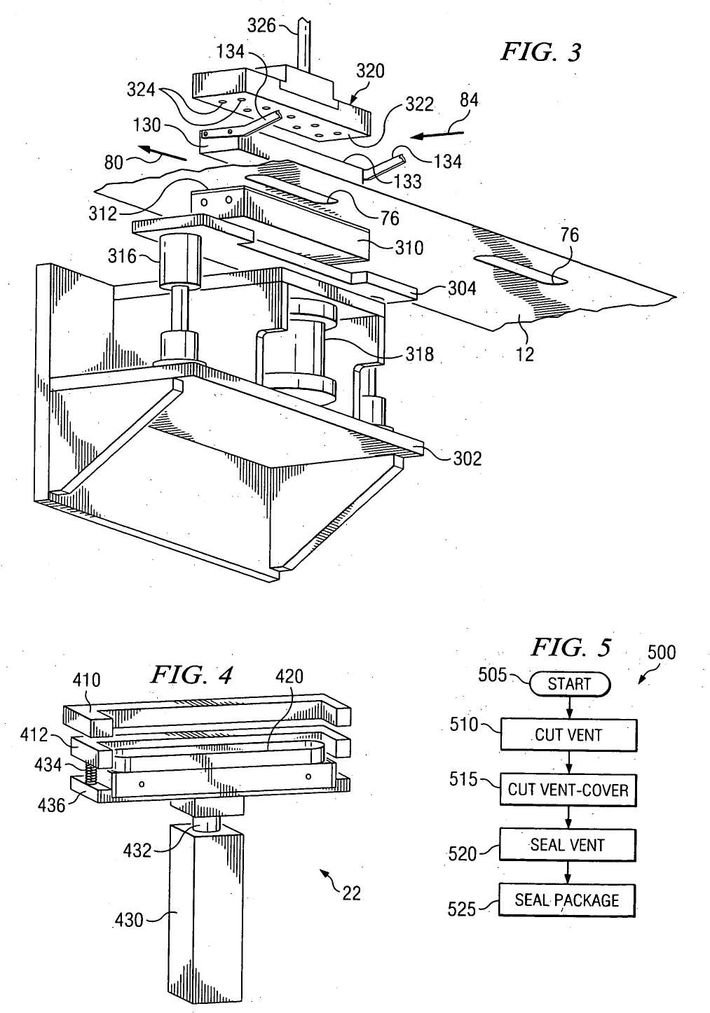

FIG. 5 illustrates a method 500 according to an implementation of the invention. At step 505, the process begins. At step 510, the vent-cutting system cuts a vent in a top-web of a thermoplastic or other suitable material. At step 515, a vent-cover system cuts a portion of the PVM web into a vent-cover for transfer to a sealing system. At step 520, the vent-cover is transferred to the sealing system, and the sealing system seals the vent-cover to the top-web to cover the vent cut at step 510. At step 525, the top-web, with the vent-cover sealed over the vent, is sealed to a bottom-web to form a package capable of sterilization through the vent-cover.

Though the method and apparatus has been described in great detail, numerous modifications and alternatives may be used in accordance with the present invention. For example, as illustrated, the transfer direction 84 is shown to be substantially normal to the production direction 82 and the vent-cutting direction 80. In an alternative implementation, the transfer direction 84 could be the same or similar to the vent-cutting direction 80 or the production direction 82. Accordingly, other embodiments are within the scope of the following claims.

1. A method for manufacturing a sterilizable package, the method comprising the steps of:
cutting a vent in a portion of a top-web;
cutting a vent-cover of porous vent media (PVM) from a PVM web, wherein the vent-cover is sized larger than the vent, the porous vent media adapted to permit sterilization there through;
sealing the vent-cover to the top-web using heat and pressure over a time period to cover the vent; and
sealing the portion of the top-web to a portion of a bottom-web using heat and pressure over a time period, wherein sealing the top-web to the bottom-web creates a sealed package perimeter, and wherein the vent-cover is within the sealed package perimeter.

2. The method of claim 1, further comprising the step of cutting the sealed portions of the top-web and bottom web from the top and bottom web to create a discrete sealed package.

3. The method of claim 1, wherein the porous vent media is a polyolefin fiber web.

4. The method of claim 1, wherein the porous vent media is a paper web.

5. The method of claim 1, wherein the porous vent media is a polyethylene web.

6. The method of claim 1, further comprising the steps of:
introducing the top-web in a first direction, wherein the top-stock comprises a roll of thermoplastic material; and
introducing the PVM web in a second direction.

7. The method of claim 6, wherein the second direction is substantially normal to the first direction.

8. The method of claim 6, wherein the second direction is substantially opposite of the first direction.

9. The method of claim 6, wherein the step of cutting the portion of PVM from the PVM web comprises cutting the vent-cover substantially normal to the second direction, wherein the vent-cover has a width measured substantially in the second direction that is about the same or less than a length measured substantially normal to the width.

10. The method of claim 9, wherein the PVM is introduced in a direction substantially normal to the length of the direction of production, the vent-cover is cut substantially parallel to the axis of production, the vent-cover having a length and a width greater than the length and width of the vent.

11. The method of claim 9, wherein the length of the vent-cover is equal to the width of the PVM web measured perpendicular to the direction of introduction, and wherein the width of the vent-cover is less than the length of the vent-cover.

12. The method of claim 9, wherein the step of cutting the vent patch includes:
extruding a lead edge of the PVM web onto a transfer block;
holding the lead edge of the PVM on the transfer block using a tamp pad; and
cutting the vent-cover from the PVM web, wherein the vent-cover comprises the portion of the PVM web remaining on the transfer block after cutting.

13. The method of claim 12, wherein the portion of the PVM web remaining on the transfer block is held on a transfer block face by vacuum pressure supplied by vacuum ports disposed on a face of the transfer block.

14. The method of claim 13, further including the steps of:
determining whether the vent-cover is in place on the transfer block face; and
biasing the tamp pad onto the vent cover to press the vent cover onto the transfer block face, the pressing of the vent-cover onto the transfer block face operable to allow the vacuum ports to exert vacuum pressure onto the vent-cover.

15. The method of claim 1, wherein the vent-cover is sealed to the top-web portion such that the vent-cover is contained within the package as defined by the top-web portion sealed to the bottom-web portion.

16. The method of claim 1, wherein the top-web is introduced opposite to a direction of production, the vent having a length and a width, wherein the length is greater than the width; the length measured substantially along the direction of production.

17. The method of claim 16, further comprising the step of transferring the vent-cover to a support brille positioned above adjacent to the vent in the top-web, wherein the support brille includes vacuum ports adapted to hold the vent-cover in place when the vent-web is transferred to the support brille.

18. The method of claim 16, further comprising the step of determining whether the vent-cover is retained on the support brille by the vacuum ports.

19. The method of claim 18, further comprising the step of automatically re-attempting to transfer the vent-cover to the support brille upon the determination that the vent-cover was not retained on the support brille.

20. The method of claim 19, wherein the step of sealing the vent includes the steps of:
placing the vent-cover in contact with the vent in the top web on a first side of the top web; and
bringing a pre-heated seal plate into contact with the top-web on a second side of the top-web opposite from the support brille and vent-cover, the seal plate providing heat and pressure over a time period to seal the vent-cover to the top-web between the seal plate and the support brille.

21. A system for manufacturing a sterilizable package, comprising:
a top-web;
a vent cutting system adapted to create a vent in the top-web;
a porous vent media (PVM) web, the PVM web adapted to allow gasses to pass there through and to substantially reduce the passage of bacteria there through;
a vent-cover cutting system adapted to remove a vent-cover from the PVM web, the removed vent-cover having dimensions slightly greater than the vent in the top-web;
a first sealing system operable to seal the vent-cover to the top-web, wherein the vent-cover is sealed around the vent in the top-web; and
a second sealing system operable to seal a portion of the top-web to a portion of a bottom-web, wherein the top-web sealed to the bottom stock web forms an enclosed space, the enclosed space bounded by the sealed portions of the top-web and bottom-web.

22. The system of claim 21, wherein the top-web comprises a thermoplastic material.

23. The system of claim 21, wherein the PVM web comprises polymer fibers.

24. The system of claim 21, wherein the vent-cover cutting system includes:
a drive motor operable to draw the PVM web into a cutting area via a system of rollers;
a transfer block operable to transfer the vent-cover to the first sealing system;
a blade adapted to remove the vent-cover from the PVM web; and
a tamp-pad adapted to maintain the vent-cover in position on the transfer block when the blade removes the vent-cover from the PVM web.

25. The system of claim 24, wherein the system of rollers includes:
a drive roller coupled to the drive motor; and
a pinch roller disposed adjacent to the drive roller and adapted to allow the PVM web to move there between due to the force applied to the PVM via the drive roller and the pinch roller.

26. The system of claim 24, wherein the first sealing system includes:
a support brille adapted to accept the vent-cover from the transfer block, the support brille having a vacuum port operable to maintain the position of the vent-cover on the support brille; and
a seal plate having a sealing face positioned opposite the vent-cover, wherein the vent in the top-web is positioned there between, wherein the sealing face is heated and operable to seal the vent-cover to the top-web when the sealing face and the vent-cover are brought in contact with the top-web, and wherein the vent-cover has sufficient dimensions to cover the vent in the top-web after vent-cover is sealed to the top-web.

27. The system of claim 26, wherein the first sealing system further comprises a vent-cover sensor adapted to detect the presence of the vent-cover on the support brille, wherein the vent-cover sensor is coupled to the support brille.

28. The system of claim 26, wherein the vent-cover sensor is further adapted to communicate the absence of the vent-cover after a first attempt to transfer the vent-cover from the transfer block to the support brille to a transfer controller, the transfer controller operable to direct the support brille and transfer to re-attempt the transfer of the vent-cover from the transfer block to the support brille.
